(19) United States
(12) Patent Application Publication
(10) Pub. No.: US 2021/0362154 A1
(43) Pub. Date: Nov. 25, 2021

(54) PARTICLE TRAPPING CHAMBER, PARTICLE TRAPPING CHIP, PARTICLE COLLECTING METHOD, AND PARTICLE SORTING DEVICE

(71) Applicant: SONY CORPORATION, TOKYO (JP)

(72) Inventors: KENSUKE KOJIMA, KANAGAWA (JP); SHIN MASUHARA, TOKYO (JP)

(21) Appl. No.: 16/979,297

(22) PCT Filed: Dec. 10, 2018
(86) PCT No.: PCT/JP2018/045233

§ 371 Date: Sep. 9, 2020

(30) Foreign Application Priority Data
Mar. 19, 2018 (JP) 2018-051228

(51) Int. Cl. B01L 3/00; B01D 29/92

(52) U.S. Cl. CPC B01L 3/502761; B01D 29/925; B01L 2300/0887; B01L 2200/0668; B01D 2201/204

(57) ABSTRACT

To provide a particle trapping chamber, a particle trapping chip, a particle collecting method, and a particle sorting device, capable of selectively collecting microparticles without directly labeling the microparticles. A particle trapping chamber includes at least a particle trapping unit having a well with a hole, and a particle trapping channel unit used for trapping a particle in the well. The hole causes the well and the particle trapping channel unit to communicate with each other, and at least one of the hole or the particle trapping unit has an inner wall coated with a thermally fusible substance.

### TECHNICAL FIELD

The present invention relates to a particle trapping chamber, a particle trapping chip, a particle collecting method, and a particle sorting device.

### BACKGROUND ART

Conventionally, in single cell analysis technology, one cell is trapped in each of a large number of microwells arranged on a plane, and the form of each cell is individually observed to analyze the characteristics of each cell or to analyze a reaction of each cell with a reagent using, for example, fluorescence and the like as an index. A channel is connected to the microwells, and a device having a microwell and a channel is called a microfluidic device.

Precise microfluidic technology is used for such a microfluidic device, and it is required to perform priming of a channel or to regulate a flow of a fluid using a valve and the like.

A valve used in the microfluidic device has also been developed. For example, Cited Document 1 discloses technology of “adding a meltable material to a microchannel, melting the meltable material by a heater, forcing the meltable material into a second channel by air pressure, and cooling and solidifying the melted material to block a flow”.

Furthermore, Cited Document 2 discloses technology of expanding a valve material contained in a chamber of a microfluidic device by a thermal coil to block a flow.

Meanwhile, examples of a microfluidic device that traps one cell in each of microwells for analyzing a single cell include a device disclosed in Patent Document 3.

In the device, a hole is formed in each of the wells, and a cell is trapped by suction through the hole. With this technology, trapping in the wells can be performed more efficiently. However, for example, in a case where a larger number of cells than the number of the wells are applied, cells that are not trapped in the wells are precipitated near the wells. The cells precipitated near the wells may have an adverse effect in a case where cells trapped in the wells are observed and/or measured or in a case where cells trapped in the wells are taken out by a device such as a micromanipulator, for example.

In order to remove the cells precipitated near the wells, it is conceivable to wash out these cells, for example. However, even if a flow for washing out these cells is formed, a flow rate is almost zero on a surface of a chip having the wells, and therefore a high flow rate at a certain degree is required in order to wash out the precipitated cells. Meanwhile, in a case where the flow rate formed in order to wash out the precipitated cells is too high, a cell trapped in a well near the precipitated cells may come out of the well or a cell trapped in the well may be damaged. As described above, it is not easy to remove cells precipitated near wells.

Furthermore, in order to solve the problems described above, it is conceivable to apply a smaller number of cells than the number of wells. However, in this case, since a flow due to suction is hardly formed around wells that have already trapped cells, cells can also be precipitated around the wells that have already trapped cells. In addition, additional cells may be precipitated in wells that have already trapped cells.

Therefore, in order to solve the problems described above, a particle trapping chamber, which is new single particle trapping technology, has been developed.

The particle trapping chamber described above includes at least

a particle trapping unit having at least one well or through hole, and a particle trapping channel unit used for trapping a particle in the well or in the through hole, and

the particle is trapped in the well or in the through hole by suction to the side opposite to a settling side of the particle through the particle trapping channel unit (Japanese Patent Application No. 2017-171921).

### CITATION LIST

### Patent Document

### Patent Document 1: PCT International Application Laid-Open No. 2005/107947

### Patent Document 2: PCT International Application Laid-Open No. 99/01688

### Patent Document 3: Japanese Patent Application Laid-Open No. 2011-163830

### SUMMARY OF THE INVENTION

### Problems to be Solved by the Invention

The trapping chamber described above performs suction to the side opposite to the settling side of the particles, traps the particles in the wells, optically marks specific particles, then applies a reverse pressure to a slit to release all the particles from the wells, and all the particles are collected. Thereafter, using a device such as flow cytometry, only the optically marked cells are sorted and collected.

However, since marking is optically performed in the wells, microparticles such as cells may be damaged, for example, and biological behaviors of the cells may change because the cells themselves are labeled.

Furthermore, since a reverse pressure is applied to the slit to collect all the particles, there is a concern that time may elapse before a subsequent sorting step.

Moreover, in consideration of a load in the sorting step and the like, index sorting in which cells are labeled and collected while being in the wells is desired.

### Solutions to Problems

In order to solve the above problems, in the present technology, by disposing a material that physically closes a slit in each well, a function capable of selectively collecting cells without directly labeling the cells is added.

That is, the present technology provides a particle trapping chamber including at least a particle trapping unit having a well with a hole, and a particle trapping channel unit used for trapping a particle in the well, in which

the hole causes the well and the particle trapping channel unit to communicate with each other, and

at least one of the hole or the well has an inner wall coated with a thermally fusible substance.

The particle is trapped in a well with the hole by suction to the side opposite to a settling side of the particle through the particle trapping channel unit.

In addition, the thermally fusible substance can be fused by light irradiation. The thermally fusible substance fused by the light irradiation can close the hole.

The hole and/or the well is preferably tapered or inversely tapered.

Furthermore, the thermally fusible substance can form at least one layer of a multilayer film formed on an inner wall of the hole and/or the well.

A lower layer of the multilayer film preferably has a light reflecting film or a near-infrared absorbing film.

Furthermore, the hole may have a crank shape.

The thermally fusible substance preferably has a melting point of about 60° C., and the thermally fusible substance can be selected from the group including a paraffin, stearic acid, and trioxotriangulene.

Furthermore, the present technology provides a particle trapping chip including at least a particle trapping unit having a well with a hole, in which the hole causes the well and the outside to communicate with each other, and the hole and/or the well has an inner wall coated with a thermally fusible substance.

Furthermore, the present technology provides a particle collecting method including:

a particle trapping step of trapping a particle in a well with a hole by applying a suction force to the side opposite to a settling side of the particle;

a thermally fusing step of fusing a thermally fusible substance coating a well containing a target particle and/or the hole by light irradiation;

a hole closing step of causing the fused thermally fusible substance to enter the hole of the well containing the target particle and hardening the thermally fusible substance; and

a target particle collecting step of settling the target particle on a settling side of the particle.

Moreover, the present technology provides a particle collecting method including:

a particle trapping step of trapping a particle in a well with a hole by applying a suction force to the side opposite to a settling side of the particle;

a thermally fusing step of fusing a thermally fusible substance coating a well containing a non-target particle by light irradiation;

a hole closing step of causing the fused thermally fusible substance to enter the hole of the well containing the non-target particle and hardening the thermally fusible substance; and

a target particle collecting step of discharging a target particle to a settling side of the particle.

The present technology also provides a particle sorting device including:

a particle trapping chamber including at least a particle trapping unit having a well with a hole, and a particle trapping channel unit used for trapping a particle in the well, the hole causing the well and the particle trapping channel unit to communicate with each other, the hole and/or the well having an inner wall coated with a thermally fusible substance;

a suction unit that performs suction through the particle trapping channel unit; and

a light irradiation unit that irradiates the thermally fusible substance coating the inner wall of the hole and/or the well with light.

The inner wall of the hole and/or the well can include a light irradiation control unit that selectively controls light irradiation to the thermally fusible substance coating the inner wall.

### Effects of the Invention

According to the present technology, a particle can be selectively collected without being directly labelled. In a case where the particle is a cell, sorting selection can be performed without damaging the cell and without changing a biological behavior of the cell.

Note that the effects described here are not necessarily limited, and may be any of the effects described in the present disclosure.

### BRIEF DESCRIPTION OF DRAWINGS

FIG. 1 is a schematic diagram illustrating an example of a particle trapping chamber and a situation of particle trapping using the chamber.

FIG. 2 is a schematic diagram illustrating an example of a particle trapping chamber and a situation of particle trapping using the chamber.

FIG. 3 is a schematic diagram illustrating an example in which an inner wall on one side of a well is coated with a paraffin.

FIG. 4 is a schematic diagram illustrating an example in which inner walls on both sides of a well are coated with a paraffin.

FIG. 5 is a schematic diagram illustrating an example in which a well is inversely tapered.

FIG. 6 is a schematic diagram illustrating an example in which a hole has a crank shape.

FIG. 7 is a schematic diagram illustrating an example in which a hole is tapered.

FIG. 8 is a schematic diagram illustrating an example in which a hole is coated with a multilayer film.

FIG. 9 is a schematic diagram illustrating a method for manufacturing a chip by LIM molding.

FIG. 10 is a drawing-substituting photograph illustrating a chip removed from a die.

FIG. 11 is a drawing-substituting photograph illustrating a chip having a through hole.

FIG. 12 is a drawing-substituting photograph illustrating a cross section of a manufactured chip.

FIG. 13 is a drawing-substituting photograph illustrating a chip obtained by subjecting a glass substrate and a ZEONOR sheet to laser perforation processing.

FIG. 14 is a drawing-substituting photograph illustrating a chip obtained by subjecting a glass substrate to picosecond laser perforation processing.

FIG. 15 is a drawing-substituting photograph illustrating a cross section of a manufactured chip.

FIG. 16 is a diagram schematically illustrating chip processing by SiO₂ photolithography.

FIG. 17 is a drawing-substituting photograph illustrating a well and a hole of a chip formed by SiO₂ photolithography.

FIG. 18 is a schematic diagram illustrating an example of a particle sorting device.

### MODE FOR CARRYING OUT THE INVENTION

Hereinafter, a preferred embodiment for carrying out the present technology will be described. Note that the embodiment described below exemplifies a representative embodiment of the present technology, and the scope of the present technology is not narrowly interpreted by the embodiment. The description will be made in the following order.

1. Configuration of particle trapping chamber

2. Thermally fusible substance

3. Embodiment

3-1. First Embodiment

3-2. Second Embodiment

3-3. Third Embodiment

3-4. Fourth Embodiment

3-5. Fifth Embodiment

3-6. Sixth Embodiment

4. Method for manufacturing particle trapping chamber

4-1. Mold transfer method

4-2. Laser perforation processing

4-3. SiO₂ photolithography

4-4. Coating of well and/or hole with film

4-4-1. Vacuum vapor deposition method/vacuum sputtering method

4-4-2. Reflow method

5. Particle trapping chip

6. Particle collecting method

6-1. Positive selection

6-2. Negative selection

7. Particle sorting device

Note that in the present technology, “particle” includes, for example, a biological microparticle such as a cell, a microorganism, or a liposome, and a synthetic particle such as a latex particle, a gel particle, or an industrial particle. Examples of the biological microparticle include a biological polymer polymer such as a chromosome constituting various cells, a liposome, mitochondria, an organelle, a nucleic acid, a protein, or a complex thereof. Examples of the cell include an animal cell (a hematocyte and the like) and a plant cell. Examples of the microorganism include bacteria such as Escherichia coli, viruses such as tobacco mosaic virus, fungi such as yeast, and the like.

Note that the size of the particle is not limited to that of a fine particle, and the present technology can be applied regardless of the size of the particle.

### 1. CONFIGURATION OF PARTICLE TRAPPING CHAMBER

A particle trapping chamber includes at least a particle trapping unit having at least one well, and a particle trapping channel unit used for trapping a particle in the well, and the particle is trapped in the well or in the through hole by suction to the side opposite to a settling side of the particle through the particle trapping channel unit. For details, refer to Japanese Patent Application No. 2017-171921.

Note that in the present technology, “well” refers to a portion defining a space in which a particle is trapped, and the shape of the well is not particularly limited as long as having a space in which a particle is trapped. Examples of the shape include an inverted recess, a through hole, a shape obtained by combining an inverted recess and a through hole, a tapered shape, an inversely tapered shape, and the like.

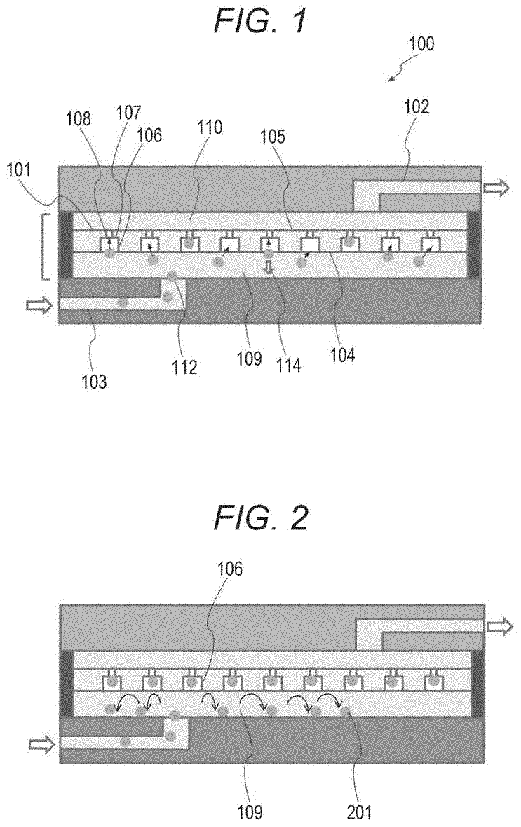

An example of the particle trapping chamber and a situation of particle trapping using the chamber will be described with reference to FIGS. 1 and 2. FIGS. 1 and 2 are each a schematic diagram illustrating an example of the particle trapping chamber of the present technology and a situation of particle trapping using the chamber.

In FIG. 1, a particle trapping chamber 100 includes a particle trapping unit 101 and a particle trapping channel unit 102, and further includes a fluid supplying channel unit 103. The particle trapping unit 101 has a particle trapping surface 104 and a surface 105 facing the side opposite to the particle trapping surface 104. The particle trapping surface 104 has a plurality of wells 106. A hole 108 is formed on a top surface portion 107 of each of the wells. The hole 108 penetrates the chip from the top surface portion 107 of the well to the surface 105 opposite to the particle trapping surface. The particle trapping chamber 100 is disposed such that gravity acts on a particle 112 in a direction of arrow 114. Each of the wells 106 has a size containing only one particle 112.

In FIG. 1, a space in the particle trapping chamber 100 is divided by the particle trapping unit 101 into a space 109 on a settling side of a particle and a space 110 on the side opposite to the space 109.

A container (not illustrated) storing a fluid containing the particle is connected to the fluid supplying channel unit 103. The fluid supplying channel unit 103 supplies the fluid containing the particle into the chamber 100. The fluid supplying channel unit 103 is connected to the space 109 on the settling side at a bottom of the chamber 100 (that is, a surface on which a particle settles). The fluid containing the particle is supplied from the container to the space 109 on the settling side through the fluid supplying channel unit 103.

Note that the fluid supplying channel unit 103 may be connected to the space 109 on the settling side in a portion other than the bottom of the chamber. For example, the fluid supplying channel unit 103 may be disposed so as to communicate with the space 109 on the settling side on a side surface of the chamber.

Suction is performed through the particle trapping channel unit 102 by a pump (not illustrated) connected to the particle trapping channel unit 102. The particle trapping channel unit 102 is connected to the space 110 on the opposite side at a ceiling of the chamber 100 (that is, the surface opposite to the surface on which a particle settles).

Note that the particle trapping channel unit 102 may be disposed in a portion other than the ceiling of the chamber. For example, the particle trapping channel unit 102 may be disposed so as to communicate with the space 110 on the opposite side on a side surface of the chamber.

By performing suction with the pump, the fluid containing the particle is supplied from the container to the space 109 on the settling side through the fluid supplying channel unit 103. By further continuing the suction, the particle 112 floats in the space 109 on the settling side and enters any one of the wells 106. The particle 112 that has entered any one of the wells 106 strikes an entrance of the hole 108 and stops moving at the entrance. This is because the size of the hole 108 is smaller than the size of the particle 112, and therefore the particle 112 cannot pass through the hole 108. In this way, the particle is trapped within the well 106.

In particle trapping using the particle trapping chamber 100 of FIG. 1, the particle 112 is guided into the well 106 by suction, and therefore a possibility that a particle will be trapped in each of the wells is increased.

Furthermore, FIG. 2 illustrates an example of movement of a particle that has not been trapped in a well. As illustrated in FIG. 2, a particle 201 that has not been trapped in a well settles on a bottom of the space 109 on a settling side by an action of gravity. As a result, the particle that has not been trapped does not stay near the well 106.

Furthermore, in a well that has trapped a particle, a hole in the well is blocked by the particle, and therefore entrance of another particle into the well is suppressed. That is, further entrance of a particle into a well that has already trapped a particle is suppressed.

A particle that has been trapped in a well can be subjected to various observations and/or measurements. For example, a predetermined fluorescent label is attached to particles before the particles are supplied into the chamber, and a particle that emits the strongest fluorescence after being trapped can be selected from the trapped particles. Moreover, only the selected particle can be taken out of the particle trapping chamber 100 with a single particle acquiring device such as a micromanipulator, for example. Then, another treatment is performed using the selected particle. In a case where the particle is a cell, the another treatment can be, for example, genetic analysis, culture, substance production, or the like.

Through the above series of operations, for example, selection of a particle having desired characteristics, such as selection of a cell that performs desired antibody secretion, selection of a cell or a microorganism that performs desired gene expression, or selection of a cell having a desired differentiation ability, is possible.

### 2. THERMALLY FUSIBLE SUBSTANCE

In the particle trapping chamber of the present technology, the hole, the well, or both the hole and the well have inner walls coated with a thermally fusible substance.

The thermally fusible substance is fused by light irradiation, and is not particularly limited as long as not affecting a fine particle such as a cell. Preferably, the thermally fusible substance is solid at room temperature, has a melting point of about 60° C., and has a low vapor pressure. Specifically, the thermally fusible substance may be a paraffin, stearic acid, trioxotriangulene, or a combination thereof.

In addition, the thermally fusible substance is more preferably a material having an absorption band in a light wavelength region with less cytotoxicity. Furthermore, preferably, the thermally fusible substance is hydrophobic and has a low specific gravity.

A paraffin is a semi-transparent to white soft solid that is not dissolved in water at room temperature and is a chemically stable substance.

Characteristics of various paraffins, such as a melting point, are illustrated in Table 1 below.

TABLE 1

| | | | | | | | | | | | Average |
|---|---|---|---|---|---|---|---|---|---|---|---|
| | | | | | | | | | | | molecular |
| | Melting | | Oil | | | Viscosity | | Density | | Flash | weight |
| | point | | content | Penetration | | mm²/s | Hue | (g/cm³) | | point | (Gas |
| Number | ° C. | (° F.) | mass % | 25° C. | 35° C. | (100° C.) | Saybolt | 25° C. | 80° C. | ° C. | chromatography) |
| 155 | 69 | (156) | 0.2 | 15 | 20 | 6.4 | +30 | 0.927 | 0.783 | 262 | 472 |
| | | | | | | | | | (80° C.) | | |
| 150 | 66 | (151) | 0.2 | 14 | 20 | 5.6 | +30 | 0.925 | 0.784 | 258 | 458 |
| 140 | 61 | (142) | 0.2 | 11 | 17 | 4.1 | +30 | 0.920 | 0.776 | 242 | 404 |
| 135 | 58 | (136) | 0.3 | 13 | 21 | 3.9 | +30 | 0.911 | 0.775 | 234 | 389 |
| 130 | 55 | (131) | 0.3 | 14 | 32 | 3.8 | +30 | 0.908 | 0.772 | 228 | 373 |
| 125 | 53 | (127) | 0.3 | 17 | 59 | 3.3 | +30 | 0.902 | 0.771 | 222 | 361 |
| 120 | 50 | (122) | 0.3 | 23 | 83 | 3.1 | +30 | 0.901 | 0.769 | 212 | 344 |
| 115 | 47 | (117) | 0.5 | 30 | 90 | 3.0 | +30 | 0.900 | 0.768 | 208 | 338 |

Preferably, the thermally fusible substance is solid at room temperature, and has a melting point of about 60° C. Therefore, as the thermally fusible substance, paraffins having the characteristics of the numbers 150, 140, 135, 130, 125, 120, and 115 in Table 1, for example, are more suitably used. In particular, the paraffin of the number 140 is preferable because of having a melting point of about 60° C.

Other examples of the thermally fusible substance include stearic acid (molecular formula C₁₇H₃₅COOH, molecular weight 284.5 g/mol, vapor pressure: 133 Pa (174° C.), melting point 69 to 72° C., flash point 196° C. specific gravity (water=1): 0.94 to 0.83, water-insoluble, white solid).

Still other examples of the thermally fusible substance include trioxotriangulene (TOT).

A derivative of trioxotriangulene is an organic neutral radical, but is as stable as an ordinary organic molecule. Furthermore, trioxotriangulene forms a one-dimensionally stacked structure in a crystal. In addition, a crystal of a trioxotriangulene derivative has an absorption band in a wavelength region of 1000 nm to 1500 nm and strongly absorbs near infrared light.

Note that as a document on trioxotriangulene, “Near-infrared absorption of n-stacking columns composed of trioxotriangulene neutral radicals”, Yasuhiro Ikabata, Qi Wang, Takeshi Yoshikawa, Akira Ueda DOI: 10.1038/s41535-017-0033-8, International Application Laid-Open No. 2010/061595 A1, and the like can be referred to.

Furthermore, a vapor deposition method is used in a step of coating a well or a hole with the thermally fusible substance, which will be described later. Examples of a reference document for vapor deposition of trioxotriangulene include Japanese Patent Application Laid-Open No. 2017-22287.

Hereinafter, each embodiment will be described by taking a paraffin as an example of the thermally fusible substance.

### 3. EMBODIMENT

### 3-1. First Embodiment

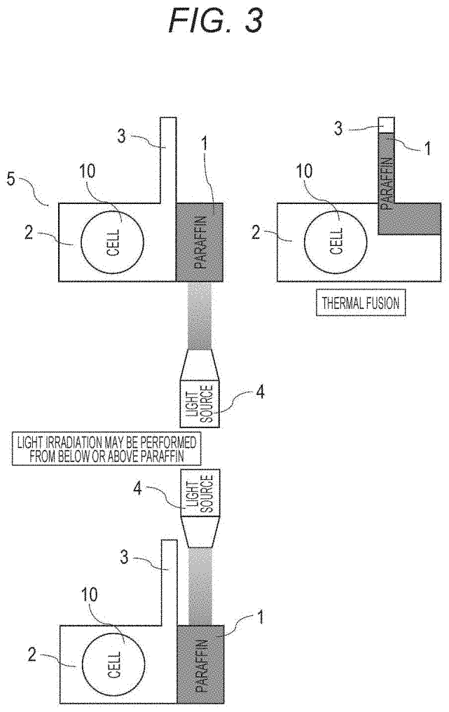

FIG. 3 illustrates an example in which an inner wall on one side of a well 2 is coated with a paraffin 1.

After a single cell 10 is trapped by a certain particle trapping unit 2, the paraffin 1 on the inner wall on one side of the well 2 is irradiated with light from a light source 4. Then, as illustrated in the right side of FIG. 3, the paraffin 1 on the inner wall on one side is thermally fused, and the paraffin 1 enters a hole 3 formed in an upper surface of the well 2 by a capillary phenomenon. The paraffin 1 in the hole 3 is hardened by natural cooling to close the hole. As a result, a suction force applied to the well 2 is stopped. Even if the paraffin 1 is melted, the hole 3 is closed by hydrophobic interaction.

At this time, the light irradiation may be performed from below or above the paraffin 1 on the inner wall on one side of the well 2 (upper and lower stages on the left side of FIG. 3). Furthermore, the paraffin 1 is preferably located at such a position that the cell 10 is not irradiated with light during light irradiation.

### 3-2. Second Embodiment

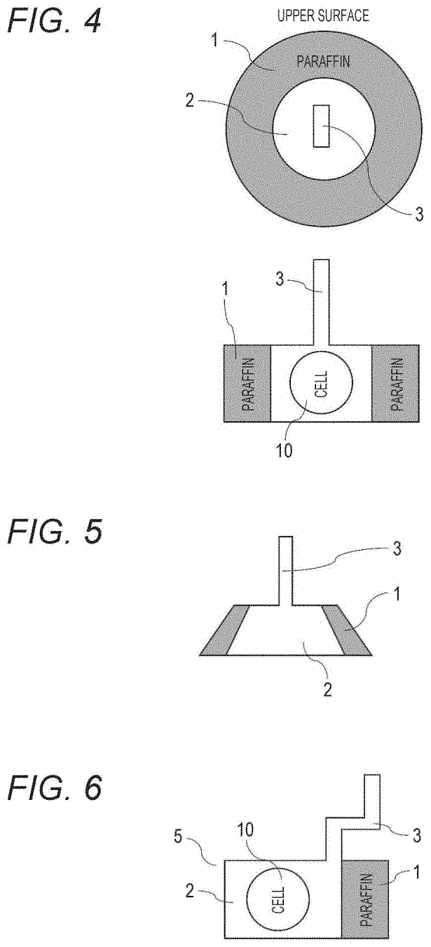

FIG. 4 illustrates an example in which inner walls on both sides of a well 2 are coated with a paraffin 1. When the well 2 is viewed from above, as illustrated on the right side of FIG. 4, a hole 3 is formed in the center of the well 2, and the well 2 is coated with the paraffin 1 such that the paraffin 1 surrounds the well 2.

When the paraffin 1 is irradiated with light and melted in the configuration of FIG. 4, the paraffin 1 enters the hole 3 at the center of an upper surface by a capillary phenomenon and is solidified by natural cooling to close the hole 3.

### 3-3. Third Embodiment

FIG. 5 illustrates an example in which a well 2 is inversely tapered. Note that the well 2 may be tapered. A hole 3 in the center of an upper surface of the well 2 is closed by the paraffin 1 in a similar manner to the second embodiment.

### 3-4. Fourth Embodiment

FIG. 6 illustrates an example in which a hole 3 has a crank shape. By having a crank shape, the hole 3 can have a cooling portion for cooling the paraffin 1 that has entered the hole 3 by a capillary phenomenon, and an embolus portion for closing the hole 3 more completely.

### 3-5. Fifth Embodiment

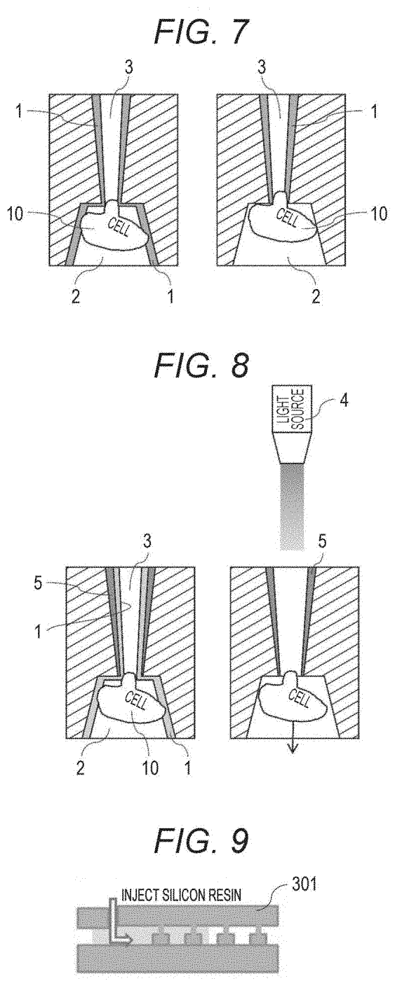

FIG. 7 illustrates an example in which a hole 3 is tapered. Note that the hole 3 may be inversely tapered.

The left side of FIG. 7 illustrates that a well 2 and the hole 3 are coated with a paraffin 1. The right side of FIG. 7 illustrates that the hole 3 is coated with the paraffin 1.

In the present technology, only the well 2, only the hole 3, or both of the well 2 and the hole 3 may be coated with the paraffin 1, but the hole 3 is thin at a joint between an upper surface of the well 2 and the hole 3. Therefore, large invasion of a cell 10 into the hole 3 can be suppressed. Furthermore, even if the cell 10 is deformed and invades the hole 3, by melting the paraffin 1 of the hole 3 in a portion invaded by the cell 10 when the cell is discharged below the well by applying a positive pressure from the hole 3 toward the well 2, the cell 10 can be discharged without being damaged.

Note that by making the well 2 or the hole 3 tapered or inversely tapered, an area coated with the paraffin 1 can be increased.

### 3-6. Sixth Embodiment

FIG. 8 illustrates an example in which a hole 3 is coated with a multilayer film.

The left side of FIG. 8 illustrates an example in which the hole 3 is coated with a reflective film 5, and then a paraffin 1 is stacked on the reflective film 5. When the reflective film is in a lower layer, the paraffin film can be melted more quickly during light irradiation from the light source 4. Furthermore, by superposing a layer on a layer, it is possible to perform fine control to reduce the diameter of the hole 3.

Alternatively, a near infrared film may be used instead of the reflective film, and the paraffin 1 can be stacked on the near infrared film. When the near infrared film is used, heat is generated by irradiation of infrared light with less cytotoxicity from the light source 4, and the paraffin 1 can be melted more quickly.

Furthermore, a well 2 may also be coated with a multilayer film. Alternatively, the well may be coated with a material that does not easily adhere to a cell.

Moreover, the multilayer is not limited to two layers, and a layer may be further stacked in order to control the size of the diameter of the well 2 or the hole 3, or a layer other than the reflective film, the near infrared film, and the film of a material that does not easily adhere to a cell may also be stacked. However, at least one layer of the multilayer preferably contains a thermally fusible substance such as a paraffin.

### 4. METHOD FOR MANUFACTURING PARTICLE TRAPPING CHAMBER

Examples of a method for manufacturing a chip having a well 2 or a hole 3 include a method for performing molding with a high-definition 3D printer, a method for molding a PDMS resin using a master die to manufacture a chip, a method for directly processing glass into a well 2 or a hole 3 with a laser, a method for manufacturing a SiO₂ membrane using a semiconductor process, and other methods.

### 4-1. Mold Transfer Method

Examples of a mold transfer method include an injection molding method by forming liquid injection molding (LIM) as illustrated in FIG. 9. A sealed die 301 to be used for forming a well 2, a hole 3, and the like is prepared. Two or more low-viscosity materials are injected into the die 301. When these materials become a polymeric plastic by a reaction between the materials, the polymeric plastic is removed from the die 301. For the LIM formation, for example, polyurethane, polyurea, polyisocyarate, polyester, polyepoxy, polyamide, or the like is used.

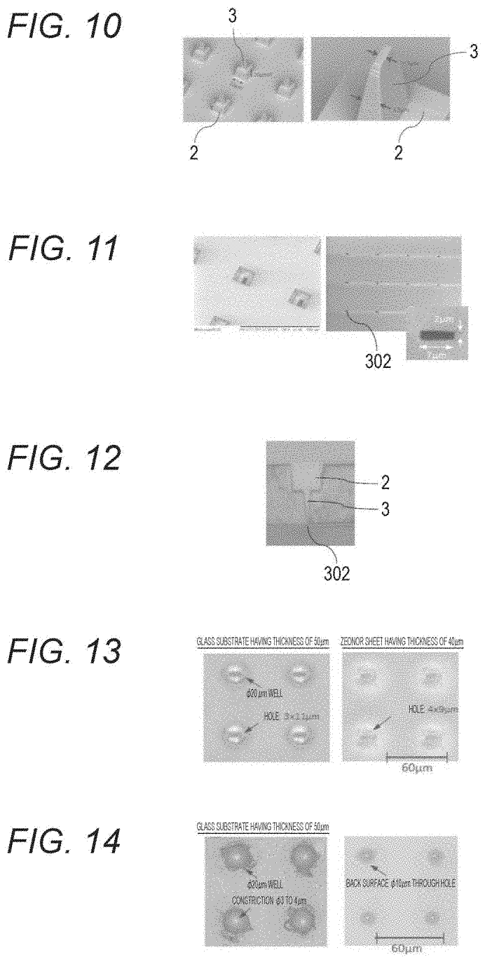

FIG. 10 illustrates an enlarged photograph of the chip removed from the die 301. Here, a square well 2 has a piece of about 22 μm and a height of about 20 μm. A hole 3 is inversely tapered and has a size of about 1 μm or less at a narrow portion and about 3.5 μm at a middle portion. The hole 3 does not penetrate the chip at this point.

A through hole of the hole 3 is formed by subjecting the hole 3 to back surface laser polishing. FIG. 11 illustrates an enlarged photograph of the chip having a through hole formed therein. As illustrated in FIG. 11, the through hole has a lateral width of about 7 μm and a longitudinal width of about 2 μm.

FIG. 12 illustrates a photograph of a cross section of the manufactured chip. It can be seen that the hole 3 is tapered from the well 2 and penetrates the chip.

### 4-2. Laser Perforation Processing

Laser perforation processing is a method for processing a resin plate or a glass plate with a laser. A commercially available laser processing machine can be used.

The left side of FIG. 13 illustrates a product obtained by subjecting a glass substrate having a thickness of 50 μm to excimer laser perforation processing. The right side of FIG. 13 illustrates a product obtained by subjecting a ZEONOR sheet having a thickness of 40 μm to excimer laser perforation processing. In the glass substrate, a well 2 having a diameter of about 20 μm was formed, and a hole 3 having a size of about 3×11 μm was formed. In the ZEONOR sheet, a hole 3 having a size of about 4×9 μm was formed. The substrates of both materials could be processed favorably. Note that processing was performed at a wavelength of 193 nm.

FIG. 14 illustrates a product obtained by subjecting a glass substrate having a thickness of 50 μm to picosecond laser perforation processing. A well 2 having a diameter of about 20 μm was formed, and a constriction having a diameter of about 3 to 4 μm, serving as a joint between the well 2 and a hole 3, was formed. A hole having a diameter of about 10 μm, serving as a through hole of the hole 3, was formed on a back surface.

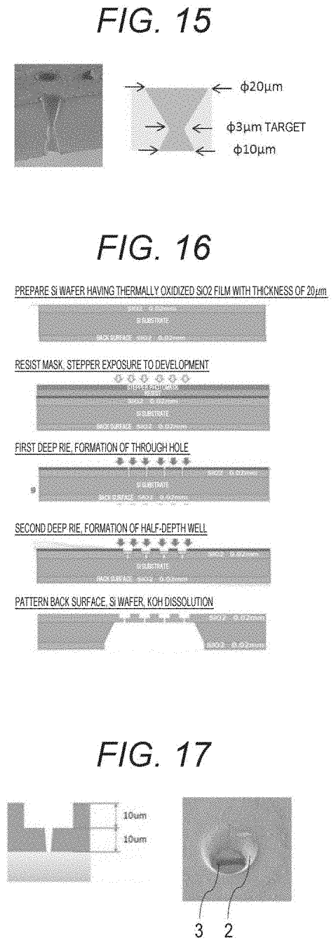

FIG. 15 illustrates a photograph of a cross section of the manufactured chip. By using the laser perforation processing, the well 2 or the hole 3 can have a gradient, and a tapered or cone-shaped well or hole can be formed.

### 4-3. SiO₂ Photolithography

There is also a method used for manufacturing a semiconductor element, in which a substrate containing silicon is subjected to fine processing by photolithography.

FIG. 16 schematically illustrates processing of a chip by SiO₂ photolithography.

Front and back surfaces of a Si substrate are coated with a thermally oxidized SiO₂ film with a thickness of 20 μm to manufacture a Si wafer. A resist mask is applied to the thermally oxidized SiO₂ film on the front surface. Projection exposure is performed in a stepping manner on a wafer, and development is performed.

Next, a first Deep RIE is performed to form a hole 3 and a through hole of the hole 3. A second Deep RIE is further performed to form a well 2. Finally, the wafer is subjected to alkaline etching (for example, KOH dissolution) from a back surface thereof to form a chip.

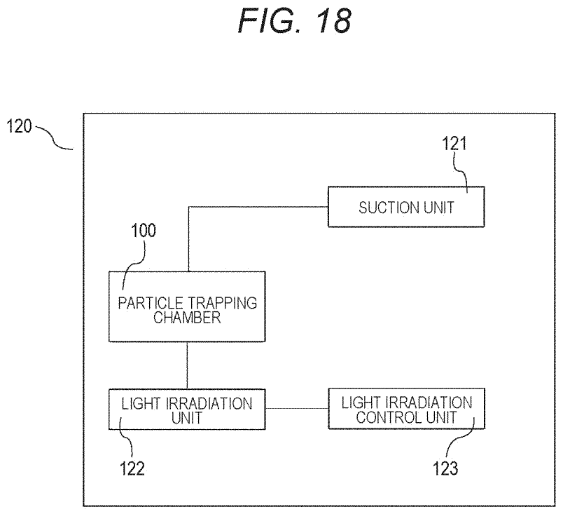

FIG. 17 illustrates the well 2 and the hole 3 of the chip formed by SiO₂ photolithography.

Note that in any of the manufacturing methods, a well 2 and/or a hole 3 is preferably processed into a tapered shape or a cone shape such that a sidewall of the well 2 or the hole 3 is easily coated with a coating material for protecting a cell, a light reflecting film, a thermally fusible substance, and the like. By forming a side surface of the well 2 or the hole 3 into a tapered shape or the like, a film having functionality such as a coating material can be easily formed on the side surface, and a multilayer film can also be formed. In terms of formation, since the fine structure of the well or the hole is continuous, it is desirable to use semiconductor process technology using vapor deposition, sputtering, and the like for the functional film. By this method, the above multilayer film is easily formed. For example, an arrangement method is possible in which a light reflecting film or the like is formed as a base and then coated with a transparent closing material of a thermally fusible substance.

In the present technology, in order to make the opening area of the joint between a well 2 and a hole 3 smaller than a processing limit, the hole 3 can be formed into an inversely tapered shape, and the side wall of the hole 3 can be subjected to multilayer coating to make the substantial area of the opening portion have a fine shape. As a result, even if a cell diameter is equal to or smaller than the processing limit size of the hole 3, the opening area of the hole 3 can be narrowed to the extent that the cell does not pass.

Furthermore, if the well 2 is tapered, it is easy to ensure a place for disposing the closing material of the thermally fusible substance at a position where light does not interfere with a cell trapped in the well 2.

### 4-4. Coating of Well and/or Hole with Film

### 4-4-1. Vacuum Vapor Deposition Method/Vacuum Sputtering Method

A metal mask is applied to the chip in which the well 2 and the hole 3 have been formed as described above. The chip is put in a vacuum vapor deposition tank, and the vacuum vapor deposition tank is sufficiently evacuated. Meanwhile, a paraffin, which is a thermally fusible substance of a vapor deposition target, is prepared in the vacuum vapor deposition tank while being placed on a vapor deposition boat containing tungsten and connected to a thermoelectric heater.

When the vacuum reaches 1E-6 Torr, an electric current is caused to flow in a heating wire to heat the tungsten board. When the paraffin starts to become a solution on the boat by being sufficiently heated, a shutter is opened and vapor deposition is started. By heating the paraffin, which is an embolus material, until the paraffin is vaporized such that a saturated vapor pressure in vacuum becomes 0.1 Torr, the vapor deposition is performed such that a desired place is completely coated while a solid matter is vaporized at a substrate temperature (room temperature). After the vapor deposition, it is confirmed with an interference color that a paraffin film is formed on an enzyme surface taken out of the vacuum tank and fixed.

### 4-4-2. Reflow Method

A solid paraffin is put in a tank, and the paraffin is fused by heating and naturally cooled to be fixed. By using a material having an absorption band in a light wavelength region with less cytotoxicity as a thermal reflow film, an influence on a cell can be reduced.

It is only required to form a multilayer film by changing a material used for coating and repeatedly using the vacuum vapor deposition method/vacuum sputtering method and a reflow method.

In particular, since the opening area of the hole 3 is defined by the processing size limit of a manufacturing method, it is difficult to process the hole 3 into a shape sufficiently smaller than a cell. After processing, a multilayer film can be formed by coating, and the opening area can be narrowed from the surroundings to control the degree of opening. Therefore, a slit opening area that is sufficiently small with respect to a cell can be arbitrarily manufactured.

Furthermore, the chip manufactured by the above method may be damaged by hangnail at the time of processing a side wall of the well 2 when a cell comes into contact with the side wall of the well 2 during cell trapping. When the cell further adheres to unevenness of the side wall of the well 2 and is held by the well 2, it is difficult to easily release the cell to the outside of the well 2 even if a reverse pressure is applied when the cell is taken out of the well 2. In order to solve this, it is important to coat and cover a side surface of the well 2. Even if the cell further adheres to a gently uneven portion on the side wall of the well 2 due to a force of hydrophobic interaction or the like, in a case where a thermally fusible coating material is disposed, by melting the coating material in an adhesion interface to form a gap by applying heat from the outside by light irradiation and the like, the cell easily flows and can be easily released from the well 2.

### 5. PARTICLE TRAPPING CHIP

A particle trapping chip of the present technology can be manufactured by the above method and may be disposable. The particle trapping chip includes a particle trapping unit having a well 2 with a hole 3, in which the hole 3 causes the well 2 and the outside to communicate with each other, and the hole and/or the well has an inner wall coated with a thermally fusible substance.

In this structure, during cell trapping, some of trapped cells may be held in the well 2 in a state where the cells are sucked and deformed in an opening of a joint between the well 2 and the hole 3. In this case, even if a reverse pressure is applied to the well 2, the cells are not easily released from the well 2. If it is tried to forcibly discharge the cells from the well 2 by applying a high pressure, the cells are highly likely to be damaged. Therefore, by irradiating the thermally fusible substance previously coated on the side surface of the hole 3 with light from the outside, the thermally fusible substance coating layer of a contact portion where a cell invades the hole 3 is thermally fused to form a gap. This makes it easy to release the cell from the hole 3, and makes it possible to release the cell from the well 2 without damage at a low reverse pressure.

As for the chip, the chip obtained by forming the particle trapping unit having the well 2 and the hole 3 by the above method, and a substrate having a channel are mounted in layers to be formed into a particle trapping chamber for use.

Moreover, a cover glass and a port jig are pressed against upper and lower end surfaces of the particle trapping chamber, and are screwed with a metal fixing jig to perform pressure sealing.

Then, the well 2, the hole 3, and the inside of the channel in the particle trapping chamber are filled with water by a priming operation to expel bubbles. A Jurkat cell or a K562 cell is introduced from a cell introduction port of the particle trapping chamber. Suction is performed at a slight pressure from a back surface of the hole 3 by a suction pump. A cell that has settled on a bottom surface of the microparticle trapping chamber is caused to float, is transported into the well 2, and is trapped. As the operation condition, for example, a suction force is set to −100 Pa.

Then, a specific assay is performed in the well 2, and a target cell is sorted.

After the cell is sorted, for example, the thermally fusible substance coated on a well 23 of the cell to be released is irradiated with light. When the thermally fusible substance is hydrophobic and has a low specific gravity, the thermally fusible substance moves upward and enters the hole 3 by a capillary phenomenon. Since the inside of the hole 3 is sufficiently thin to the extent that a cell does not pass through the hole 3, the thermally fusible substance stays in the tube due to hydrophobic interaction and is naturally cooled and hardened. This makes it possible to close the hole 3.

### 6. PARTICLE COLLECTING METHOD

In the particle trapping chamber, verification was performed by using a cell as a microparticle. As a result, it was confirmed by experiments and simulations that a cell was trapped in the well even if a suction pressure of the hole 3 was sufficiently weak.

However, when the suction pressure of the hole 3 is completely stopped, the cell settles on a bottom surface by its own weight. By using this, sorting of positive selection or negative selection can be performed.

### 6-1. Positive Selection

Positive selection is

a particle collecting method including:

a particle trapping step of trapping a particle in a well with a hole by applying a suction force to the side opposite to a settling side of the particle;

a thermally fusing step of fusing a thermally fusible substance coating a well containing a target particle and/or a hole by light irradiation;

a hole closing step of causing the fused thermally fusible substance to enter the hole of the well containing the target particle and hardening the thermally fusible substance; and

a target particle collecting step of settling the target particle on a settling side of the particle.

That is, the hole 3 of the well 2 that has trapped a particle that is desired to be collected is closed by light irradiation. Then, the suction pressure of the hole 3 is stopped, and the particle spontaneously falls from the well 3. By performing indexing and light irradiation in the order in which particles are desired to be collected, that is, in the order in which the particles are dropped, particles can be collected in that order.

### 6-2. Negative Selection

Negative selection is

a particle collecting method including:

a particle trapping step of trapping a particle in a well with a hole by applying a suction force to the side opposite to a settling side of the particle;

a thermally fusing step of fusing a thermally fusible substance coating a well containing a non-target particle and/or a hole by light irradiation;

a hole closing step of causing the fused thermally fusible substance to enter the hole of the well containing the non-target particle and hardening the thermally fusible substance; and

a target particle collecting step of discharging a target particle to a settling side of the particle.

That is, a hole 3 of a well 2 containing a particle other than the target particle is closed by light irradiation. From the closed well 2, a particle cannot be expelled. Therefore, only a target particle can be collected by expelling.

### 7. PARTICLE SORTING DEVICE

FIG. 18 illustrates an example of a particle sorting device.

A particle sorting device 120 of the present technology includes:

the particle trapping chamber 100 including at least a particle trapping unit having a well with a hole, and a particle trapping channel unit used for trapping a particle in the well, the hole causing the well and the particle trapping channel unit to communicate with each other, the hole and/or the well having an inner wall coated with a thermally fusible substance;

a suction unit 121 that performs suction through the particle trapping channel unit; and

a light irradiation unit 122 that irradiates the thermally fusible substance coating the inner wall of the well and/or the hole with light.

The light irradiation unit 122 may include a light irradiation control unit 123 that selectively controls light irradiation to the thermally fusible substance coating the inner wall of the well and/or the hole.

The light irradiation control unit 123 can appropriately select to close a hole 3 of a well 2 that has trapped a target particle or to close a hole 3 of a well 2 that has trapped a particle other than the target particle.

Furthermore, although not illustrated, the particle sorting device 120 can include: a fluid control unit that controls a flow of a liquid; a particle detection unit that detects presence or absence of a particle trapped in a well; an analysis unit that analyzes a particle trapped in a well; a storage unit that records analysis data and the like; a display unit that displays the state of a well, analysis data, and the like; an input unit through which a user operates action of the particle sorting device; and the like.

Note that the present technology can have the following configurations.

[1]

A particle trapping chamber including at least:

a particle trapping unit having a well with a hole; and

a particle trapping channel unit used for trapping a particle in the well, in which

the hole causes the well and the particle trapping channel unit to communicate with each other, and

at least one of the hole or the well has an inner wall coated with a thermally fusible substance.

[2]

The particle trapping chamber according to [1], in which the particle is trapped in a well with the hole by suction to the side opposite to a settling side of the particle through the particle trapping channel unit.

[3]

The particle trapping chamber according to [1] or [2], in which the thermally fusible substance is fused by light irradiation.

[4]

The particle trapping chamber according to [3], in which the thermally fusible substance fused by the light irradiation closes the hole.

[5]

The particle trapping chamber according to any one of [1] to [4], in which the hole and/or the well is tapered or inversely tapered.

[6]

The particle trapping chamber according to any one of [1] to [5], in which the thermally fusible substance forms at least one layer of a multilayer film formed on the inner wall of the well and/or the hole.

[7]

The particle trapping chamber according to [6], having a light reflecting film or a near-infrared absorbing film in a lower layer of the multilayer film.

[8]

The particle trapping chamber according to any one of [1] to [7], in which the hole has a crank shape.

[9]

The particle trapping chamber according to any one of [1] to [8], in which the thermally fusible substance has a melting point of about 60° C.

[10]

The particle trapping chamber according to any one of [1] to [9], in which the thermally fusible substance is selected from the group including a paraffin, stearic acid, and trioxotriangulene.

[11]

A particle trapping chip including at least a particle trapping unit having a well with a hole, in which the hole causes the well and the outside to communicate with each other, and the hole and/or the well has an inner wall coated with a thermally fusible substance.

[12]

A particle collecting method including:

a particle trapping step of trapping a particle in a well with a hole by applying a suction force to the side opposite to a settling side of the particle;

a thermally fusing step of fusing a thermally fusible substance coating a well containing a target particle and/or a hole by light irradiation;

a hole closing step of causing the fused thermally fusible substance to enter the hole of the well containing the target particle and hardening the thermally fusible substance; and

a target particle collecting step of settling the target particle on a settling side of the particle.

[13]

A particle collecting method including:

a particle trapping step of trapping a particle in a well with a hole by applying a suction force to the side opposite to a settling side of the particle;

a thermally fusing step of fusing a thermally fusible substance coating a well containing a non-target particle and/or a hole by light irradiation;

a hole closing step of causing the fused thermally fusible substance to enter the hole of the well containing the non-target particle and hardening the thermally fusible substance; and

a target particle collecting step of discharging a target particle to a settling side of the particle.

[14]

A particle sorting device including:

a particle trapping chamber including at least a particle trapping unit having a well with a hole, and a particle trapping channel unit used for trapping a particle in the well, the hole causing the well and the particle trapping channel unit to communicate with each other, the hole and/or the well having an inner wall coated with a thermally fusible substance;

a suction unit that performs suction through the particle trapping channel unit; and

a light irradiation unit that irradiates the thermally fusible substance coating the inner wall of the well and/or the hole with light.

[15]

The particle sorting device according to [14], further including a light irradiation control unit that selectively controls light irradiation to the thermally fusible substance coating the inner wall of the well and/or the hole.

### REFERENCE SIGNS LIST

1 Paraffin
2 Well
3 Hole
4 Light source
10 Single cell
100 Particle trapping chamber
101 Particle trapping unit
102 Particle trapping channel unit
103 Fluid supplying channel unit
106 Well
108 Hole
120 Particle sorting device
121 Suction unit
122 Light irradiation unit
123 Light irradiation control unit

1. A particle trapping chamber comprising at least:
a particle trapping unit having a well with a hole; and
a particle trapping channel unit used for trapping a particle in the well, wherein
the hole causes the well and the particle trapping channel unit to communicate with each other, and
at least one of the hole or the well has an inner wall coated with a thermally fusible substance.

2. The particle trapping chamber according to claim 1, wherein the particle is trapped in a well with the hole by suction to a side opposite to a settling side of the particle through the particle trapping channel unit.

3. The particle trapping chamber according to claim 1, wherein the thermally fusible substance is fused by light irradiation.

4. The particle trapping chamber according to claim 3, wherein the thermally fusible substance fused by the light irradiation closes the hole.

5. The particle trapping chamber according to claim 1, wherein the hole and/or the well is tapered or inversely tapered.

6. The particle trapping chamber according to claim 1, wherein the thermally fusible substance forms at least one layer of a multilayer film formed on the inner wall of the well and/or the hole.

7. The particle trapping chamber according to claim 6, having a light reflecting film or a near-infrared absorbing film in a lower layer of the multilayer film.

8. The particle trapping chamber according to claim 1, wherein the hole has a crank shape.

9. The particle trapping chamber according to claim 1, wherein the thermally fusible substance has a melting point of about 60° C.

10. The particle trapping chamber according to claim 1, wherein the thermally fusible substance is selected from the group including a paraffin, stearic acid, and trioxotriangulene.

11. A particle trapping chip comprising at least a particle trapping unit having a well with a hole, wherein the hole causes the well and an outside to communicate with each other, and the hole and/or the well has an inner wall coated with a thermally fusible substance.

12. A particle collecting method comprising:
a particle trapping step of trapping a particle in a well with a hole by applying a suction force to a side opposite to a settling side of the particle;
a thermally fusing step of fusing a thermally fusible substance coating a well containing a target particle and/or the hole by light irradiation;
a hole closing step of causing the fused thermally fusible substance to enter the hole of the well containing the target particle and hardening the thermally fusible substance; and
a target particle collecting step of settling the target particle on a settling side of the particle.

13. A particle collecting method comprising:
a particle trapping step of trapping a particle in a well with a hole by applying a suction force to a side opposite to a settling side of the particle;
a thermally fusing step of fusing a thermally fusible substance coating a well containing a non-target particle and/or the hole by light irradiation;
a hole closing step of causing the fused thermally fusible substance to enter the hole of the well containing the non-target particle and hardening the thermally fusible substance; and
a target particle collecting step of discharging a target particle to a settling side of the particle.

14. A particle sorting device comprising:
a particle trapping chamber including at least a particle trapping unit having a well with a hole, and a particle trapping channel unit used for trapping a particle in the well, the hole causing the well and the particle trapping channel unit to communicate with each other, the hole and/or the well having an inner wall coated with a thermally fusible substance;
a suction unit that performs suction through the particle trapping channel unit; and
a light irradiation unit that irradiates the thermally fusible substance coating the inner wall of the hole and/or the well with light.

15. The particle sorting device according to claim 14, further comprising a light irradiation control unit that selectively controls light irradiation to the thermally fusible substance coating the inner wall of the hole and/or the well.
